(19) United States
(12) Patent Application Publication
(10) Pub. No.: US 2009/0162345 A1
(43) Pub. Date: Jun. 25, 2009

(54) Materials and Methods for Reversing Type-1 Diabetes

(76) Inventors: Mark A. Atkinson, Gainesville, FL (US); Gregory Simon, Gainesville, FL (US); Clive Henry Wasserfall, Gainesville, FL (US); Abraham Scaria, Cambridge, MA (US); Desmond A. Schatz, Gainesville, FL (US); Donna Armentano, Cambridge, MA (US); Srinivas Shankara, Cambridge, MA (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/094,866

(22) PCT Filed: Nov. 29, 2006
(86) PCT No.: PCT/US06/45786

§ 371 Date: Oct. 20, 2008

Related U.S. Application Data
(60) Provisional application No. 60/751,617, filed on Nov. 29, 2005.
(60) Provisional application No. 60/816,659, filed on Jun. 27, 2006.

(51) Int. Cl. A61K 39/395; A61P 3/10

(52) U.S. Cl. 424/130.1

(57) ABSTRACT

In accordance with the subject invention, anti-thymocyte globulin (ATG) can be used to modulate a patient's immune response in order to prevent and/or delay the onset or the progression of type 1 diabetes. ATG treatment augments CD4+CD25+ cell frequencies and their functional activities.

### BACKGROUND OF THE INVENTION

Diabetes mellitus is a family of disorders characterized by chronic hyperglycemia and the development of long-term vascular complications. This family of disorders includes type 1 diabetes, type 2 diabetes, gestational diabetes, and other types of diabetes.

Immune-mediated (type 1) diabetes (or insulin dependant diabetes mellitus, IDDM) is a disease of children and adults for which there currently is no adequate means for treatment or prevention. Type 1 diabetes, represents approximately 10% of all human diabetes. The disease is characterized by an initial leukocyte infiltration into the pancreas that eventually leads to inflammatory lesions within islets, a process called “insulitis”.

Type 1 diabetes is distinct from non-insulin dependent diabetes (NIDDM) in that only the type 1 form involves specific destruction of the insulin producing beta cells of the islets of Langerhans. The destruction of beta cells appears to be a result of specific autoimmune attack, in which the patient's own immune system recognizes and destroys the beta cells, but not the surrounding alpha cells (glucagon producing) or delta cells (somatostatin producing) that comprise the pancreatic islet. The progressive loss of pancreatic beta cells results in insufficient insulin production and, thus, impaired glucose metabolism with attendant complications.

Type 1 diabetes is currently managed by the administration of exogenous human recombinant insulin. Although insulin administration is effective in achieving some level of euglycemia in most patients, it does not prevent the long-term complications of the disease including ketosis and damage to small blood vessels, which may affect eyesight, kidney function, blood pressure and can cause circulatory system complications.

The potential for islet or pancreas transplantation has been investigated as a means for permanent insulin replacement. This approach, though initially attracting much interest, has been severely hampered by the difficulties associated with obtaining sufficient quantities of tissue, as well as the relatively low rate at which transplanted islets survive and successfully graft the recipient. Other potential treatments using a variety of agents to reverse type 1 diabetes, even in the absence of cell or whole organ transplantation, have also been disappointing.

The factors responsible for type 1 diabetes are complex and thought to involve a combination of genetic, environmental, and immunologic influences that contribute to the inability to provide adequate insulin secretion to regulate glycemia.

The natural history of type 1 diabetes prior to clinical presentation has been extensively studied in search of clues to the etiology and pathogenesis of beta cell destruction. The prediabetic period may span only a few months (e.g., in very young children) to years (e.g., older children and adults). The earliest evidence of beta cell autoimmunity is the appearance of various islet autoantibodies. Metabolically, the first signs of abnormality can be observed through intravenous glucose tolerance testing (IVGTT). Later in the natural history of the disease, the oral glucose tolerance test (OGTT) typically becomes abnormal. With continued beta cell destruction and frank insulinopenia, type 1 diabetes becomes manifest.

Type 1 diabetes occurs predominantly in genetically predisposed persons. Concordance for type 1 diabetes in identical twins is 30-50% with an even higher rate of concordance for beta cell autoimmunity, as evidenced by the presence of islet autoantibodies in these individuals (Pyke, D. A., 1979. “Diabetes: the genetic connections.” Diabetologia 17: 333-343). While these data support a major genetic component in the etiopathogenesis of type 1 diabetes, environmental or non-germline genetic factors must also play important pathologic roles. Environmental factors proposed to date include viral infections, diet (e.g., nitrosamines in smoked meat, infant cereal exposure), childhood vaccines, breast-feeding, and early exposure to cows' milk. Hence, while the list of potential environmental agents for type 1 diabetes is large, the specific environmental trigger(s) that precipitate beta cell autoimmunity remain elusive.

A growing body of evidence suggests that failure to regulate the immune response plays a major role in the pathogenesis of type 1 diabetes (You, S. et al. “Autoimmune Diabetes Onset Results from Qualitative Rather than Quantitative Age-Dependent Changes in Pathogenic T-Cells,” Diabetes. 2005, 54: 1415-1422). In terms of the cellular basis for this immunoregulatory failure, patients with (or rodent models of) type 1 diabetes have potential deficiencies in at least two regulatory T cell populations, NKT cells and CD4+CD25+ T cells (Lederman, M. M. et al. J. Immunol. 1981, 127: 2051-2055; Asano, M. et al. J. Exp. Med. 1996, 184:387-396; Salomon, B. et al. Immunity. 2000, 12:431-440; Wu, A. J. et al. Proc. Natl. Acad. Sci. U.S.A. 2002, 99: 12287-12292). In addition to defects in regulation, developmental and functional defects have also been reported in the antigen-presenting cells of both NOD mice (an animal model of type 1 diabetes) and human type 1 diabetes patients; including those of differentiation and function of macrophages and dendritic cells (DC) (Liu, J. et al. 2002. J. Immunol. 169:581-586; Serreze, D. V. et al 1993. J. Immunol. 150:2534-2543; Alleva, D. G. et al. 2000. Diabetes 49:1106-1115; Dahlen, E. et al. 2000. J. Immunol. 164:2444-2456; Kukreja, A. et al. 2002. J. Clin. Invest. 109: 131-140; Weaver, D. J. et al. 2001. J. Immunol. 167:1461-1468; Yan, G. et al. 2003. J. Immunol. 170:620-627).

Indeed, since the findings of Sakaguchi reporting multi-organ autoimmunity of mice subjected to thymectomy in early life (Sakaguchi, S. 2004. Annu. Rev. Immunol 22:531-562), other studies have suggested. that CD4+CD25+ regulatory T cells function as major regulators of the immune response and impact the development of autoimmunity. CD4+CD25+ T cells comprise approximately 5-10% of the peripheral CD4+ T cell population in mice and humans. CD4+CD25+ T cells do not by their nature proliferate in vitro (i.e, anergic) to antigenic stimulation, their suppressive properties require functional activation by antigenic stimulation, and the strength of that signal combined with the degree of costimulation all affect the degree of regulator function (Takahashi, T. et al. 1998. Int. Immunol. 10:1969-1980; Thornton, A. M. et al. J. Immunol. 2000, 164:183-190).

Baecher-Allan et al. have reported that only cells expressing a high level of CD25 correlate with the regulatory functions ascribed to the CD4+CD25+ regulatory T cells described in mice (2001. J. Immunol. 167:1245-1253). A majority of studies suggest they control so called “effector T cell” (Teff) proliferation in vitro through direct cell-cell interaction, while transforming growth factor β (TGF-β) and other cytokines may also be involved in these processes (Takahashi, T. et al. 1998. Int. Immunol. 10:1969-1980; Stephens, L. A. et al. 2001. Eur. J. Immunol. 31:1247-1254; Thornton, A. M. et al. 1998. J. Exp. Med. 188:287-296).

Many intracellular, surface expressed, or secreted molecules have been reported as being involved in the development and/or maintenance of CD4+CD25+ regulatory T cells. Examples include (but are not limited to) IL-2, CD28/B7, CTLA-4, STAT-5a, ICOS/ICOSL, OX-40/OX-40L, and CD40/CD40L (Baecher-Allan, C. et al. 2004 Semin. Immunol. 16:89-98; Suzuki, H. et al. 1995 Science 268:1472-1476; Malek, T. R. et al. 2000 J. Immunol. 164:2905-2914; Wolf, M. et al. 2001 Eur. J. Immunol. 31:1637-1645; Kagami, S. et al. 2001 Blood 97:2358-2365; Takeda, I. et al. 2004 J. Immunol 172:3580-3589; Kumanogoh, A. et al. 2001. J. Immunol. 166:353-360). Studies of mice suggest that TGF-β can induce the conversion of CD4+CD25− cells into CD4+CD25+ regulatory cells in vitro (Chen, W. et al. 2003. J. Exp. Med. 198:1875-1886).

Whereas an association between the fork head transcription factor (Foxp3) expression and the acquisition of a regulatory T cell phenotype has been reported, very little information exists as to the mechanisms underlying the regulation of Foxp3 or its transcriptional targets (Fontenot, J. D. et al., 2005 “Regulatory T Cell Linage Specification by the Forkhead Transcription Factor Foxp3,” Immunity, 22: 329-341).

One key modulator of regulatory T cell function may be TGF-β, as evidenced by the observation that TGF-β can induce Foxp3 expression on human CD4+ T-cells in vitro (Horwitz, D. A. et al. 2003. J. Leukoc. Biol. 74:471-478). However, it remains uncertain as to the mechanism by which TGF-β induces Foxp3, if at all (Nakamura, K. et al. 2001. J. Exp. Med. 194:629-644; Piccirillo, C. A. et al. 2002. J. Exp. Med. 196:237-246).

Analysis of CD4+CD25+ T cells in various autoimmune prone animals have in most cases implied that such animals have an intrinsic defect in either the frequency or function of their regulatory T cells. Furthermore, such defects have often been related to actual disease development. In studies of NOD mice wherein the goal was to compare the frequency of CD4+CD25+ T cells against other common inbred strains, most (but not all) suggested NOD mice express a relative deficiency in these regulatory T cells (Gombert, J. M. et al. 1996 Eur. J. Immunol. 26:2989-2998). Furthermore, these cells were capable of imparting disease protection.

As far as a role for CD4+CD25+ regulatory T cells in human autoimmune disease, subjects with multiple sclerosis and autoimmune polyglandular syndrome type II (APSII) have both been described to have normal levels of CD4+CD25+ T cells, but impaired suppressive function of these regulatory cells (Viglietta, V. et al. 2004. J. Exp. Med. 199:971-979; Kriegel, M. A. et al. 2004. J. Exp. Med. 199:1285-1291). In contrast, subjects with SLE have been reported to have reduced frequencies of CD4+CD25+ T cells (Liu, M. F. et al. 2004. Scand. J. Immunol. 59:198-202). In terms of type 1 diabetes, subjects with this disorder demonstrate abnormal functional activity, characterized by decreased suppressive activity of CD4+CD25+ T cells and abnormal cytokine production (Brusko, T. et al, Diabetes, 2005, 54:1407-1414).

While recent studies of anti-CD3 therapy have generated a marked degree of enthusiasm based on their efficacy for T1D reversal in both NOD mice and humans (Herold, K. C., Hagopian, W., Auger, J. A., Poumian-Ruiz, E., Taylor, L., Donaldson, D., Gitelman, S. E., Harlan, D. M., Xu, D., Zivin, R. A., et al. 2002. Anti-CD3 monoclonal antibody in new-onset type 1 diabetes mellitus. N Engl J Med 346:1692-1698; Keymeulen, B., Vandemeulebroucke, B., Ziegler, A. G., Mathieu, C., Kaufman, L., Hale, G., Gorus, F., Goldman, M., Walter, M., Candon, S., et al. 2005. Insulin needs after CD3-antibody therapy in new-onset type 1 diabetes. N Engl J Med 352:2598-2608), in the late 1970's, studies were reported describing the ability for another antibody based reagent, anti-lymphocyte serum (ALS), to reverse T1D in BB rats (Like, A. A., Rossini, A. A., Guberski, D. L., Appel, M. C., and Williams, R. M. 1979. Spontaneous diabetes mellitus: reversal and prevention in the BB/W rat with antiserum to rat lymphocytes. Science 206:1421-1423). More recently, ALS has been observed as an effective means to reverse T1D in NOD mice, especially when used in combination with the glucagon-like peptide-I like molecule, exendin-4; a molecule having the potential to induce β cell regeneration in mice Like, A. A., Rossini, A. A., Guberski, D. L., Appel, M. C., and Williams, R. M. 1979. Spontaneous diabetes mellitus: reversal and prevention in the BB/W rat with antiserum to rat lymphocytes. Science 206:1421-1423; Maki, T., Ichikawa, T., Blanco, R., and Porter, J. 1992. Long-term abrogation of autoimmune diabetes in nonobese diabetic mice by immunotherapy with anti-lymphocyte serum. Proc Natl Acad Sci USA 89:3434-3438). Those latter studies, while positive in terms of reporting a beneficial therapeutic outcome were, however, limited in terms of their mechanistic descriptions and lacked definition of therapeutic benefits in the natural history of T1D.

Maki and colleagues demonstrated (1992. Proc. Natl. Acad. Sci. U.S.A. 89:3434-3438) that treatment of overtly diabetic NOD mice with ALS, a polyclonal anti-T-cell antibody, induced a long-term abrogation of autoimmunity and in 50% of treated mice, achieved a lasting clinical remission. Reversal of hyperglycemia, however, was a slow process, requiring 75-105 days. Follow-up studies utilized the addition of Exendin-4, a long-acting agonist of GLP-1a potent intestinal insulinotropic hormone that augments insulin secretion in rodents as well as in both type 1 and type 2 diabetic subjects. Both GLP-1 and exendin-4 have been shown to promote replication and differentiation of beta cells in vivo (Xu, G. et al. 1999 Diabetes 48:2270-2276; Tourrel, C. et al. 2001 Diabetes 50:1562-1570) and in vitro (Zhou, J. et al. 1999 Diabetes 48:2358-2366).

In follow-up studies (Ogawa, N. et al. 2004 Diabetes 48:2358-2366), treatment of new-onset diabetic NOD mice with a combination of ALS and exendin-4 achieved complete remission in 90% of the mice. Although treatment of diabetic mice with ALS alone also achieved reversal of overt diabetes, the frequency of remission was much lower and progression to remission tended to be slower compared with ALS and exendin-4 treatment. Both untreated mice and mice treated with exendin-4 alone failed to produce any disease remission.

Other agents (non-GLP-l agonists) may be beneficial to these processes. APIDRA™ (insulin glulisine [rDNA origin]) is a human insulin analog having a rapid-acting, parenteral blood glucose lowering effect. Insulin glulisine differs from human insulin in that the amino acid asparagine at position B3 is replaced by lysine and the lysine in position B29 is replaced by glutamic acid. The glucose lowering activities of APIDRA™ and of regular human insulin are equipotent when administered by the intravenous route. After subcutaneous administration, the effect of APIDRA is more rapid in onset and of shorter duration compared to regular human insulin.

Insulin receptor substrate (IRS)-2 has been, implicated in the promotion of beta cell survival. Rakatzi et al. (2003 Biochem. Biophys. Res. Commun. 310:852-859) recently tested the hypothesis that glulisine, could mediate an enhanced beta cell protective effect due to its unique property of preferential IRS-2 phosphorylation. Specifically, these investigators monitored IRS activation by glulisine and its anti-apoptotic activity evaluated against cytokine or palmitic acid induced apoptosis on INS-1 cells in comparison to insulin, other insulin analogs, and insulin-like growth factor (IGF)-I. Glulisine induced a prominent IRS-2 activation without significant IRS-1 stimulation. The marked cytokine- and fatty acid-induced apoptosis. was strongly (5560%) inhibited by glulisine both at the level of caspase 3 activation and nucleosomal release, with only 15% inhibition of apoptosis afforded by regular insulin. At 1 nM, insulin, insulin aspart, and insulin lispro were much less effective compared to glulisine.

Anti-thymocyte globulin (ATG), has long been known to deplete lymphocytes in vivo and can effectively be used in a variety of therapeutic settings including renal transplantation, graft versus host disease, and aplastic anemia.(Smith, J. M., Nemeth, T. L., and McDonald, R. A. 2003. Current immunosuppressive agents: efficacy, side effects, and utilization. Pediatr Clin North Am 50:1283-1300, Nashan, B. 2005. Antibody induction therapy in renal transplant patients receiving calcineurin-inhibitor immunosuppressive regimens: a comparative review. BioDrugs 19:39-46; Bevans, M. F., and Shalabi, R. A. 2004. Management of patients receiving antithymocyte globulin for aplastic anemia and myelodysplastic syndrome. Clin J Oncol Nurs 8:377-382; Bacigalupo, A. 2005. Antithymocyte globulin for prevention of graft-versus-host disease. Curr Opin Hematol 12:457-462). ATG affects a wide range of immune system cells and contains antibodies against many cell surface molecules. The rapid lymphocytopenia induced by ATG in vivo has classically been attributed to several mechanisms including complement-dependent cytolysis, cell-mediated antibody-dependent cytolysis, as well as opsonization and subsequent phagocytosis by macrophages (Bonnefoy-Berard, N., Genestier, L., Flacher, M., Rouault, J.P., Lizard, G., Mutin, M., and Revillard, J.P. 1994. Apoptosis induced by polyclonal antilymphocyte globulins in human B-cell lines. Blood 83:1051-1059). It has also been suggested that ATG recognizes and cross-links multiple cell surface receptors and co-stimulatory molecules on T lymphocytes, leading to weakened T cell activation and anergy (Merion, R. M., Howell, T., and Bromberg, J. S. 1998. Partial T-cell activation and anergy induction by polyclonal antithymocyte globulin. Transplantation 65:1481-1489).

Several studies have demonstrated that ATG affects a wide range of immune cell types, having antibodies reactive with an extensive number of cell surface molecules. For example, experiments by Michallet et. al.(Michallet, M. C., Preville, X., Flacher, M., Fournel, S., Genestier, L., and Revillard, J.P. 2003, Functional antibodies to leukocyte adhesion molecules in antithymocyte globulins Transplantation 75:657-662; and Michallet, M. C., Saltel, F., Preville, X., Flacher, M., Revillard, J. P., and Genestier, L. 2003, Cathepsin-B-dependent apoptosis triggered by antithymocyte globulins: a novel mechanism of T-cell depletion. Blood 102:3719-3726.) demonstrated that ATG contains functional antibodies to CD11a/CD18 (leukocyte function-associated antigen-1 [LFA-1]) which down-modulates cell surface expression of this β2 integrin on lymphocytes, monocytes, and neutrophils. Those studies also indicated that ATG contains antibodies specific to the #1 integrin CD49d/CD29 (VLA-4), α4β7 integrin, CD50, CD54, and CD102, but not to CD62L. Binding of ATG has been observed to numerous B-lymphocyte surface proteins including CD30, CD38, CD95, CD80, and HLA-DR (Zand, M. S., Vo, T;, Huggins, J., Felgar, R., Liesveld, J., Pellegrin, T., Bozorgzadeh, A., Sanz, I., and Briggs, B. J. 2005. Polyclonal rabbit antithymocyte globulin triggers B-cell and plasma cell apoptosis by multiple pathways. Transplantation 79:1507-1515). ATG has also been shown to bind and interfere with various DC functions (Monti, P., Allavena, P., Di Carlo, V., and Piemonti, L. 2003. Effects of anti-lymphocytes and anti-thymocytes globulin on human dendritic cells. Int Immunopharmacol 3:189-196). ATG acts on DC, at least in part, by recognizing CD1a, MHC I, MHC II, CD11a, CD86, CD32, CD11b, CD29, and CD51/61. In mixed lymphocyte assays, ATG was demonstrated to inhibit T-cell proliferation by binding on T lymphocytes but not against DC, implying ATG affects DC activation but not proliferation.

Although knowledge of the immune system has become much more extensive in recent years, the precise etiology of type 1 diabetes remains a mystery. Furthermore, despite the enormously deleterious health and economic consequences, and the extensive research effort, there currently is no effective means for controlling the formation of this disease.

### BRIEF SUMMARY

The subject invention pertains to the use of anti-thymocyte globulin (ATG) in the prevention of type-I diabetes.

Advantageously, in accordance with the subject invention, ATG can be administered to a patient prior to the clinical manifestation of type 1 diabetes thereby preventing or delaying the onset of overt disease. In this regard, sufficient beta cell mass exists in certain cases near the time of symptomatic onset such that intervention with ATG, as described herein, enables the patient to retain pancreatic insulin production thereby eliminating or reducing the need for insulin injections.

In a further embodiment, administration of ATG is accompanied by administration of a compound that promotes repair, production, and/or regeneration of beta cells. The agent that promotes the repair, production, and/or regeneration of beta cells may be, for example glulisine, glucagons such as glucagon-like peptide-1 (GLP-1), DPP4 inhibitors, islet regeneration molecules, anti-apoptotic molecules and exendin-4.

### BRIEF DESCRIPTION OF THE DRAWINGS

FIG. 1 shows mATG administration diminishes the frequency of peripheral blood lymphocytes in vivo and does so with equivalent efficacy in strains both prone and non-prone to T1D. (A) 12 week old NOD or Balb/c mice (5 per group) were treated on day 0 and 3 with 1.0 mg/animal of mATG (i.e., two 500 μg/animal injections at time 0 and 72 h; noted with arrows). Whole blood was collected from tail veins and subjected to automated determination of lymphocyte counts. Shown are the lymphocyte counts±SEM. *P<0.02 in comparison to pre-administration lymphocyte counts by ANOVA. (B-D) mATG treatment transiently depletes CD3⁺, CD4⁺, and CD8⁺ T lymphocyte populations in vivo. Following the administration of mATG or rIgG into 12 week old NOD mice (1.0 mg/animal; two 500 ug doses 72 h apart; 3 per group), the frequency of specific cell populations in peripheral blood at various points in time were determined by flow cytometry, including assessment of markers for (B) CD3⁺; (C) CD4⁺; and (D) CD8⁺ cells. *P<0.01 and ** P<0.001 for comparison of the frequency of this cell population in mATG versus rIgG treated animals. (E) mATG (1.0 mg/animal; two 500 μg/animal injections at time 0 and 72 h) induces transient increases in serum IL-2 in vivo. Following administration of 1.0 mg/animal of rIgG or mATG (two 500 ug doses 72 h apart) into 12 week old NOD mice, serum samples were collected (three per group) at 0, 1, 3, 6, 12 h, as well as 1, 3, 7, 14, and 30 d. Samples were subjected to multi-plex analysis for 21 cytokines including, as shown here, IL-2. *P<0.05 and ** P<0.02 for comparison of the serum concentration of IL-2 in mATG versus rIgG treated animals.

FIG. 2 shows the development of T1D in NOD mice is prevented or reversed by mATG in a time dependent manner. 1.0 mg/animal (two 500 μg doses 72 h apart) of mATG or rIgG was administered to NOD mice (time noted by arrows). Panels represent life-tables for T1D progression, defined by the onset of overt hyperglycemia, in animals (9 per group) provided mATG or rIgG at (A) 4 week; (13) 8 week; or (C) 12 week of age. *P<0.004, by Kaplan-Meier analysis, on the rate of T1D in mATG (solid line) versus rIgG (dashed line) treated mice. For studies of disease remission, NOD mice were provided (D) rIgG or (E) mATG at their onset of overt diabetes and monitored for 4-6 week with no exogenous insulin replacement therapy. Blood glucose levels (non-fasting) in the normal range are shaded in grey.

FIG. 3 shows treatment of NOD mice with mATG attenuates insulitis over time. Following administration of 1.0 mg/animal of rIgG or mATG (two 500 ug doses 72 h apart) into 12 week old NOD mice, animals were sacrificed at 7, 14, and 30 days (3 animals per treatment group for each time). At sacrifice, pancreases were harvested, processed for hematoxylin and eosin staining, and subjected to blinded evaluation for the intensity of insulitis. In terms of assessment, samples were subjected to systematic scoring as previously described.(40) (A) In brief, (stage 0) normal islet architecture, devoid of lymphocytes; (stage 1) peri-insulitis only; (stage 2) insulitis involving <50% of the islet in cross section; and (stage 3) insulitis involving >50% of the islet; (×400). (13) Histogram depicting percentage of normal islets (stage 0, unfilled bar), peri-insulitis (stage 1, light gray bar), insulitis involving <50% of the islet in cross section (stage 2, dark gray bar), or insulitis involving >50% of the islet (stage 3, black bar). A total of 222 islets obtained from 18 animals were evaluated. The frequency of stage 0 insulitis was significantly higher in mATG treated animals than in the rIgG group (P<0.02), and, inversely, stage 3 insulitis was significantly higher in rIgG than in mATG treated animals (P<0.02).

FIG. 4 shows metabolic responses to intraperitoneal glucose challenge are improved by treatment with mATG. 30 d following treatment with rIgG or mATG, a random sampling (3 per group) of 4, 8, and 12 week old NOD mice were fasted for 5 h and subjected to intraperitoneal glucose tolerance testing (1 mg/gm body weight in saline). Blood glucose values were obtained at 0, 5, 15, 30, 60 and 120 min post-injection. Area under the curve (AUC) analysis (P<0.05), as well as determination of peak glucose levels (P<0.02), revealed an improved metabolic response to glucose stimulation in mATG versus rIgG treated animals. No differences were observed between 4 or 8 week old mice treated with rIgG or mATG (P=NS). *P<0.02.

FIG. 5 shows the distribution of antigen presenting cells is modulated in vivo by mATG treatment. 12 week old NOD mice were administrated with rIgG or mATG, at 24 h, and a variety of organs including the spleen and PLN harvested for subsequent flow cytometric analysis of resident populations. Cell surface markers (as noted) for a variety of DC, B-lymphocyte, and macrophage makers were utilized, including: (A) CD11c⁺ DC, (13) B220⁺CD45R⁺ B Lymphocytes, and (C) CD11b⁺F4/80⁺ macrophages. Flow cytometric staining for *P<0.01 in mATG vs rIgG treated animals.

FIG. 6 shows in a time dependent fashion, mATG enhances the Treg suppression of Teff in vivo. 30 d following treatment with rIgG or mATG, a random sampling (3 per group) of 4, 8, and 12 week old NOD mice were scarified and their splenocytes subjected to a purification scheme for CD4⁺CD25⁻ (Teff) and CD4⁺CD25⁺ (Treg) cells. Six replicates wells containing 1.0×10⁵ total cells per well (in the presence of irradiated accessory cells) were used in each of the following Treg:Teff ratios: 2:1, 1:1, and 0.5:1. Cells were stimulated with the combination of anti-CD3 antibody and anti-CD28 antibodies, with subsequent determination of H3 incorporation. Suppression assay for (A) 4 week, (B) 8 week, and (C) 12 week old treated mice.

FIG. 7 shows treatment with mATG modulates the diabetogenic capacity of NOD mice in vivo. (A) Adoptive transfer of 2.0×10⁶ splenocytes from 30 week old mATG or rIgG mice transferred into NOD.rag^{-/-} mice. (B) Adoptive co-transfer of 1.0×10⁶ splenocytes from 30 week old mATG or rIgG surviving mice with 1.0×10⁶ splenocytes from T1D mice transferred into NOD.rag^{-/-} mice.

### DETAILED DISCLOSURE

In accordance with the subject invention, anti-thymocyte globulin (ATG) can be used to modulate a patient's immune response in order to prevent and/or delay the onset of type 1 diabetes. One important aspect of the subject invention is the identification of a preferred therapeutic window for administering ATG to a patient.

Advantageously, in accordance with the subject invention, ATG can be administered to a patient prior to the clinical manifestation of type 1 diabetes thereby preventing or delaying the onset of overt disease. In this regard, sufficient beta cell mass exists in certain cases near the time of symptomatic onset such that intervention with ATG, as described herein, enables the patient to retain pancreatic insulin production thereby eliminating or reducing the need for insulin injections.

In a further embodiment, administration of ATG is accompanied by administration of a compound that promotes repair, production, preservation and/or regeneration of beta cells. The agent that promotes the repair, production, preservation and/or regeneration of beta cells may be, for example glulisine, glucagons such as glucagon-like peptide-l (GLP-1), DPP4 inhibitors, islet regeneration molecules, anti-apoptotic molecules and exendin-4.

ATG is an infusion of rabbit-derived antibodies against human T cells that has been used in the past for the prevention and treatment of acute rejection in organ transplantation and therapy of aplastic anemia. ATG is available, for example, from Genzyme under the trademark of Thymoglobulin®.

Specifically exemplified herein is the use of ATG to enhance the ability of CD4+CD25+ T cells (and/or other immune system cells) to defend against pathological autoimmune processes. The use of ATG in the treatment and/or prevention of type 1 diabetes is specifically exemplified herein; however, the use of ATG to reduce other pathological autoimmune conditions is contemplated according to the subject invention. Other autoimmune conditions to which the treatments of the subject invention may be applied include, but are not limited to, rheumatoid arthritis, multiple sclerosis, thyroiditis, inflammatory bowel disease, Addison's disease, pancreas transplantation, kidney transplantation, islet transplantation, heart transplantation, lung transplantation, and liver transplantation. Of particular interest according to the subject invention is the use of ATG to treat autoimmune diseases that can be improved through enhanced functionality of CD4+CD25+ T cells.

As described herein, murine ATG (mATG), in an age dependent fashion, provides intervention capable of inhibiting the development of autoimmune T1D in NOD mice. In terms of the mechanisms underlying this protection, it appears that ATG can protect β cells from autoimmune destruction via two pathways. First, a transient reduction of lymphocytes was observed in mATG treated animals. This form of immunosuppression helps prevent immune mediated disorders such as T1D. However, a second and particularly novel mechanism for ATG has been found, that being the induction of enhanced immunoregulation, defined by in vitro and in vivo enhancements of the functional activities of CD4⁺CD25⁺ T cells.

With mATG administration, lymphocyte depletion was both rapid and transient; 24 hours post administration the effects were profound but by 14 days post mATG administration, a substantial recovery in lymphocyte numbers had already occurred. The depletion and subsequent recovery appeared somewhat “non-specific” in that depletions were observed in CD3⁺, CD4⁺ and CD8⁺ T cell populations. mATG administration also induced, a marked increase in serum cytokine concentrations.

With mATG provided to NOD mice at 12 week of age, a significant reduction in insulitis occurred that maintained and even improved at 30 days post mATG administration. There were no such reductions in insulitis in groups given mATG at 4 and 8 weeks of age. The clinical benefit of reduced insulitis was reflected in overall T1D-free survival of mice (i.e, nearly 90%) that received mATG at 12 week of age. Furthermore, physiological assessment of β cell activity in response to glucose challenge 30 d post mATG administration demonstrated that even in the absence of T1D, mice treated with mATG at 12 week of age exhibited significantly lower glucose levels following metabolic stimulation when compared to control rIgG recipients. This was not the case with recipients of mATG at 4 and 8 weeks of age. The mATG mediated inhibition of T1D onset was age-dependent and dependent on the stage in the natural history of T1D development, suggesting that an active protective mechanism was induced by mATG; one which was also susceptible to inactivation at early age (i.e., 4 and 8 week of age). Cells with CD4^{+CD}25⁺ phenotype are considered to be critical in the regulation of self-tolerance and control of autoimmunity (Dejaco, C., Duftner, C., Grubeck-Loebenstein, B., and Schirmer, M. 2006. Unbalance of regulatory T cells in human autoimmune diseases. Immunology 117:289-300). Reduced levels of CD4⁺CD25⁺ T cells in both NOD mice and humans with T1D have been reported (Bevans, M. F., and Shalabi, R. A. 2004. Management of patients receiving antithymocyte globulin for aplastic anemia and myelodysplastic syndrome. Clin J Oncol Nurs 8:377-382), although the notion in both species has been controversial. Our analysis of spleens showed significant increases in CD4⁺CD25⁺ cells on d 7 and 14 post mATG injection in the 12 week old mATG treated group, as well as an enhancement of their functional activity (i.e., Treg suppression of Teff responses in vivo). This analysis also revealed increased numbers of APC in the spleen and PLN, including DC, which in certain activation states have been shown to induce Treg. While this increase may simply reflect the relative decrease in T cell number following mATG treatment, such an environment may promote the generation and/or maintenance of Treg.

Additional evidence in favor of Treg cells as the protective mechanism induced by mATG derived from in vitro immunosuppression assays. These assays, performed 30 days post mATG injection, indicated that in 12 week treated animals (unlike 4 and 8 week treated mice), recipients showed significantly higher suppression indices at varying Treg:Teff ratios (i.e., CD4⁺CD25⁺ cells: CD4⁺CD25⁻ cells). Although CD4⁺CD25⁺ Treg percentages returned to near basal levels by FACS analysis at 30 days post mATG administration, inhibition assays demonstrated retention of functional competence of these cells in the spleen. The return of Treg levels to near normal levels at 30 d post mATG treatment may be due to trafficking of these cells from spleen to pancreatic lymph nodes or to insulitis area within the pancreas. Finally, adoptive transfer of spleen cells obtained from surviving 30 week old mice from 12 week mATG group clearly demonstrated their inability to induce T1D in recipient NOD.rag^{-/-} mice, and hence attenuation of pro-diabetogenic Teff cells within this population. The adoptive co-transfer experiments directly demonstrated functionally active nature of Treg cells induced by mATG treatment (“infectious tolerance”).

In contrast to treatment with depleting anti-T cell antibodies (e.g., anti-CD4 and anti-CD8) where disease prevention depends on maintenance of T-cell depletion, a short course of ATG can establish long-term tolerance and confer permanent protection from T1D.

A further aspect of the subject invention is the use of ATG to promote treatment of disease through enhanced Foxp3 expression. Thus, the materials and methods of the subject invention can be used in the treatment of conditions including, but not limited to, type 1 diabetes, rheumatoid arthritis, multiple sclerosis, thyroiditis, inflammatory bowel disease, Addison's disease, pancreas transplantation, kidney transplantation, islet transplantation, heart transplantation, lung transplantation, and liver transplantation.

As more fully set forth herein, CD4+CD25+ T regulatory cells have been purified, their suppressive function analyzed, and their expression of Foxp3 determined. The capability of ATG to induce anti-diabetic effects and the capability for this agent to induce regulatory function and Foxp3 expression have been identified.

In a specific embodiment, 2 injections (0.5 mg) of ATG at 12 weeks of age in the NOD mouse has been found to prevent the onset of type 1 diabetes. This age in the NOD mouse represents a time just before onset of overt disease. The administration of ATG to a patient prior to the onset of clinical symptoms to prevent diabetes is a novel treatment for prevention of type 1 diabetes.

### Timing of Treatment

In a preferred embodiment, ATG is administered prior to the onset of clinical manifestation of overt type 1 diabetes. More specifically, ATG is administered at a point in disease progression when the pathological autoimmune process can be reduced by an enhanced functionality of CD4+CD25+ cells and/or by enhanced expression of Foxp3. The time of administration would also be preferably before extensive irreversible beta cell destruction as evidenced by for example, the clinical onset of type 1 diabetes. Thus, one aspect of the subject invention is the identification of a preferred point in disease progression for the administration of ATG to increase its beneficial effects in the prevention, treatment and/or reversal of pathological autoimmune response.

As set forth in more detail below with respect to type 1 diabetes, those skilled in the art, having the benefit of the instant disclosure can utilize diagnostic assays to assess the stage of disease progression in a patient and then administer ATG at the appropriate time as set forth herein.

The ability to detect susceptibility to autoimmune conditions and/or identify individuals at pre-clinical stages of the condition has improved significantly in recent years. Because of this improved ability to detect autoimmune disease at an early stage is now possible, in accordance with the subject invention, to administer ATG prior to the appearance of clinical manifestation of the disease.

With regard to the early detection of type 1 diabetes, numerous autoantibodies have been detected that are present at the onset of type 1 diabetes. Also, new serologic markers associated with type 1 diabetes continue to be described. Four islet autoantibodies appear to be the most useful markers of type 1 diabetes: islet cell antibodies (ICA), insulin autoantibodies (IAA), glutamic acid decarboxylase autoantibodies (GADA), and insulinoma-associated-2 autoantibodies (IA-2A). These are discussed in more detail below; however, the use of these markers to identify those at risk for developing type 1 diabetes is well known to those skilled in the art. In a specific embodiment of the subject invention, ATG is administered when a patient has at least one antibody marker or, preferably, at least two of the antibody markers.

ICA serve an important role as serologic markers of beta-cell autoimmunity. Seventy percent or more of Caucasians are ICA-positive at onset of type 1 diabetes. Following diagnosis, ICA frequency decreases, and fewer than 10% of patients still express ICA after 10 years. The general population frequency of ICA is between 0.1% and 0.3%. In a preferred embodiment of the subject invention, ATG is. administered prior to a decrease in ICA.

To date, insulin is the only beta-cell-specific autoantigen. IAA occur in 35-60% of children at onset of type 1 diabetes but are less common in adults. For example, in Australians with new-onset type 1 diabetes, IAA were present in 90% of children less than 5 years old, in 71% of 5-10-year-olds, and in 50% of 10-15-year-olds. In Britons with type 1 diabetes, IAA were identified in 83% of children less than 10 years old and in 56% of children 10 years old and greater.

IAA have been detected in several other autoimmune diseases. IAA were identified in 15.9% of patients with Hashimoto's thyroiditis and 13.5% of Graves' disease subjects. In another study, IAA frequencies in various thyroid autoimmune diseases were 44% in Graves' disease, 21% in primary hypothyroidism, and 23% in chronic autoimmune thyroiditis, compared with 40% in primary adrenal failure, 36% in chronic hepatitis, 40% in pernicious anemia, 25% in rheumatoid arthritis, and 29% in systemic lupus erythematosus.

Approximately 2-3% of the general population express GAD autoantibodies. These antibodies are detected in 60% or more of new-onset cases of type 1 diabetes. The IA-2A and IA-2βA general population frequencies are similar to GADA at 2-3%. IA-2A and IA-2βA are observed in 60% or more of new-onset type 1 diabetes cases.

Early biochemical evidence of beta cell injury is a decreased first-phase insulin response to the administration of intravenous glucose (IVGTT). First-phase response is defined as the insulin concentrations at +1 and +3 min following completion of an intravenous bolus injection of glucose (e.g., 0.5 g/kg). There is also a dissociation in beta cell response to secretagogues: Initially the insulin response to intravenous amino acid administration (e.g., arginine) is preserved even while first-phase responses are deficient (Ganda, O. P. et al., 1984. “differential sensitivity to beta-cell secretagogues in early, type 1 diabetes mellitus,” Diabetes 33: 516-521). In ICA-positive individuals eventually developing insulin-dependent diabetes, first-phase insulin release diminishes at a rate of about 20-40 μU/mL/year (Srikanta, S. 1984. “Pre-type 1 diabetes, linear loss of beta cell response to intravenous glucose,” Diabetes 33: 717-720).

When beta cell mass has substantially declined to less than 50% but more than 10% of normal, the OGTT may display abnormalities such as impaired fasting glucose (110-125 mg/dL) or impaired glucose tolerance (2-h glucose post-75-g challenge: 140-199 mg/dL). An abnormal OGTT prior to the clinical onset of type 1 diabetes is more likely observed in younger children. Frank clinical diabetes usually follows within 1-2 years of the onset of oral glucose intolerance. By the time acute symptoms of type 1 diabetes develop, beta cell mass is believed to have declined by approximately 90% or more from baseline. In one embodiment of the subject invention, ATG is administered once oral glucose intolerance is observed.

Most current procedures for the prediction of type 1 diabetes involve analyses of multiple islet autoantibodies. In every such study reported, nondiabetic individuals who express combinations of islet autoantibodies are found to be at greater risk for type 1 diabetes than individuals who express fewer varieties of islet autoantibodies. In addition, the total number of types of islet autoantibodies is usually more important than the specific combination of islet autoantibodies. In type 1 diabetes subjects, islet autoantibodies can also reappear after pancreas or islet transplantation, predicting failure to become insulin-independent (Bosi, E. et al. 2001. Diabetes 50:2464-2471).

Thus, in genetically predisposed individuals, an environmental trigger or triggers are believed to initiate beta cell autoimmunity, which can be identified by the presence of islet autoantibodies. With progressive beta cell damage, there is loss of first-phase insulin response to intravenous glucose administration. Subsequently the OGTT becomes abnormal, followed by symptoms of diabetes and the diagnosis of type 1 diabetes. Clearly the detection of islet autoimmunity can therefore be used as a predictive marker for the subsequent development of type 1 diabetes.

Both in nondiabetic relatives of type 1 diabetes subjects and in the general population, the detection of islet autoantibodies identifies individuals who are at high risk to develop subsequent type 1 diabetes (LaGasse, J. M. et al. 2002. Diabetes Care 25:505-511). Higher titers of ICA are more predictive than lower titers, and multiple islet autoantibodies are more powerful predictors than the presence of single autoantibodies. The combination of ICA plus low first-phase insulin secretion is possibly the strongest confirmed predictor of subsequent type 1 diabetes as demonstrated in the DPT-1. When using single autoantibodies, comparative sensitivities for the prediction of type 1 diabetes are as follows: ICA>GADA>IA-2A>>IAA. Combination islet autoantibody assays (e.g., the simultaneous detection of GADA and IA-2A (Sacks, D. B. et al. 2001. J. Clin. Chem. 47:803-804; Kawasaki, E. et al. 2000. Front Biosci. 5:E181-E190) will likely supersede ICA testing in future testing programs.

The majority of individuals with type 1 diabetes have islet autoantibodies at the time of onset of the disease. In cases where it is difficult to differentiate type 1 from type 2 diabetes, the presence of one or more islet autoantibodies (e.g., ICA, IAA, GADA, or IA-2A) is diagnostic of type 1a, immune-mediated diabetes (Rubinstein, P. et al. 1981. Hum. Immunol. 3:271-275). When individuals clinically present with a subtle, non-gketotic form of diabetes that may not be insulin-requiring yet are islet autoantibody-positive, LADA is diagnosed.

### Combination Therapy

In one embodiment, ATG can be administered with one or more additional compounds that promote beta cell regeneration and/or repair. In one embodiment, the compound that promotes regeneration and/or repair of beta cells is glulisine. Glulisine is a recombinant insulin analog that has been shown to be equipotent to human insulin. One unit of glulisine has the same glucose-lowering effect as one unit of regular human insulin. Glulisine, as is known in the art, can be administered by subcutaneous injection. After subcutaneous administration, it has a more rapid onset and shorter duration of action.

Another compound that can be administered to promote beta cell regeneration, repair and/or functionality is glucagon-like peptide-1 (GLP-1). Glucagon-like peptide and GLP derivatives are intestinal hormones that generally simulate insulin secretion during hyperglycemia, suppresses glucagons secretion, stimulate pro) insulin biosynthesis and decelerate gastric emptying and acid secretion. Some GLPs and GLP derivatives promote glucose uptake by cells but do not stimulate insulin expression as disclosed in U.S. Pat. No. 5,574,008 which is hereby incorporated by reference.

The GLP-1 used according to the subject invention may be GLP-1 (7-36), GLP-1 (7-37) or GLP-1 (1-37), or variants thereof. GLP-1 is rapidly metabolized by a peptidase (dipeptidylpeptidase IV or DPP-IV). One way to counter the rapid degradation of the hormone is to couple it to a fatty acid. Liraglutide is such a preparation. Liraglutide binds to serum albumin and is a poor substrate for the peptidase. Single injections of liraglutide give therapeutically active blood levels for 8 to 15 hours.

In a further embodiment, ATG can be administered with a GLP-1 agonist and/or GLP-1 receptor agonist. This agonist compound may be, for example, GPL-1 or exendin-4. Another GLP-1R agonist is Liraglutide. Other gut hormones that promote proliferation of islet beta cells can also be used as can compounds that activate epidermal growth factor receptor (EGFR) and the cyclic AMP-dependent transcription factor CREB.

Exendin-4 has a longer half-life than GLP-1 and has recently been shown to have a hypoglycemic effect in humans when given twice a day for one month. Exenatide is a 39-amino acid peptide which closely resembles exendin-4. It is DPP4 resistant and has many of the actions of GLP-1. That is, it slows stomach emptying, increases satiety and decreases food intake and leads to increased release and synthesis of insulin.

Other compounds that can be delivered with ATG include those that prevent or reduce β cell apoptosis. Vitamin D and prolastin are but two of these examples.

ATG may also be administered in conjunction with islet transplantation, as well as stem cell treatments and/or treatments that promote conversion of cells into insulin-secreting cells.

A further aspect of the subject invention is the use of ATG to improve the functioning of Treg cells. By modulating the function of these cells once early biochemical markers associated with an autoimmune disease are detected it is possible to delay or prevent the onset of clinical manifestations of the disease. Such use of ATG in the proper temporal therapeutic window is exemplified herein with respect to diabetes; however, the teachings set forth herein can also be readily applied to other autoimmune conditions.

Following are examples which illustrate procedures for practicing the invention. These examples should not be construed as limiting. All percentages are by weight and all solvent mixture proportions are by volume unless otherwise noted.

### Materials and Methods

Mice. Female NOD, NOD.rag^{-/-}, and Balb/c mice were purchased from Jackson Labs (Bar Harbor, Mass.), housed in specific pathogen-free facilities at the University of Florida, and provided autoclaved water and food ad libitum. NOD mice were monitored 2-3 times per week for blood glucose values indicating hyperglycemia, with T1D defined as two consecutive non-fasting blood glucose levels≧250 mg/dl separated by 24 h.

mATG administration. mATG was prepared by immunizing rabbits with pooled lymph node cells prepared from NOD, C3H/He, DBA/2, and C57BL/6 mice (Genzyme Corporation, Framingham, Mass.). Tests for quality control and quality assurance for functional activities were performed in accordance with standard procedures by the manufacturer. For studies of T1D prevention, at 4, 8, or 12 week in age, 12-18 female NOD mice (per group) were provided intraperitoneal injections of 5000 μg mATG or 500 kg rIgG (Jackson Immunologicals, Milpitas, Calif.) diluted into 200 μL saline. After 72 h, a second dose of 500 μg of either mATG or rIgG was once again administered, bringing a total dose to 1.0 mg per animal. To provide for mechanistic analysis, mice were randomly selected and sacrificed from each group for investigations 7, 14, or 28 d following mATG or rIgG administration. Using an identical dosing schedule, a separate set of studies were performed utilizing NOD mice newly-diagnosed with T1D. In these efforts, following two consecutive blood glucose readings above 250 mg/dL over 24 h, mice were provided mATG or rIgG. Animals were monitored 2-3 times per week for up to 12 week, with no exogenous insulin treatment.

Immunohistochemistry. Insulitis scoring was performed on hematoxylin and eosin stained pancreatic sections, while pancreas and spleen were stained for B220⁺ and CD3⁺ expression, as previously described (Goudy, K. S., Burkhardt, B. R., Wasserfall, C., Song, S., Campbell-Thompson, M. L., Brusko, T., Powers, M. A., Clare-Salzler, M. J., Sobel, E. S., Ellis, T. M., et al. 2003. Systemic overexpression of IL-10 induces CD4+CD25+ cell populations in vivo and ameliorates type 1 diabetes in nonobese diabetic mice in a dose-dependent fashion. J Immunol 171:2270-2278).

Lymphocyte counting and serum cytokine determination. NOD and Balb/c mice treated with mATG or rIgG were bled via tail perforation at selected times (0, 1, 3, 7, 14, and 30 d) post-injection for determination of lymphocyte counts. Samples were subjected to automated calculation using a MASCOT Hemavet 850 CBC Analyzer (Drew Scientific, Dallas, Tex.). An additional 20 μL of blood was collected from mATG or rIgG treated NOD mice at 0, 1, 3, 5, 12 hr as well as previously indicated times, and resulting serum subjected for cytokine analysis IL-1α, IL-1 β, IL-2, IL-4, IL-5, IL-6, IL-7, IL-9, IL-10, IL-12p70, IL-13, IL-15, IL-17, IFN-γ, TNF-α, GMCSF, MIP-1α, MCP-1, KC, RANTES, IP-10, and G-CSF using the Lincloplex® platform (Linco, St. Louis, Mo.), as previously described (Goudy, K. S., Burkhardt, B. R., Wasserfall, C., Song, S., Campbell-Thompson, M. L., Brusko, T., Powers, M. A., Clare-Salzler, M. J., Sobel, E. S., Ellis, T. M., et al. 2003. Systemic overexpression of IL-10 induces CD4+CD25+ cell populations in vivo and ameliorates type 1 diabetes in nonobese diabetic mice in a dose-dependent fashion. J Immunol 171:2270-2278).

Glucose tolerance testing. In 4, 8, and 12 week old NOD mice subjected to mATG or rIgG treatment, 30 days following the first injection, animals underwent intraperitoneal glucose tolerance testing. Following 5 hours of fasting, glucose (1 mg/gm body weight) was provided by intraperitoneal injection in 200 ul of saline. Blood glucose values were obtained at 0, 5, 15, 30, 60 and 120 minutes using a OneTouch Ultra® (LifeScan, Milpitas, Calif.) meter.

CD4⁺CD25⁺ T lymphocyte suppression assay. CD4⁺CD25⁺ cells were purified using a MACS® (Miltenyi Biotec, Auburn, Calif.) magnetic bead purification system, and mixed in 96 well tissue culture plates at varying ratios with CD4⁺CD25⁻ Teff lymphocytes, as previously described (Goudy, K. S., Burkhardt, B. R., Wasserfall, C., Song, S., Campbell-Thompson, M. L., Brusko, T., Powers, M. A., Clare-Salzler, M. J., Sobel, E. S., Ellis, T. M., et al. 2003. Systemic overexpression of IL-10 induces CD4+CD25+ cell populations in vivo and ameliorates type 1 diabetes in nonobese diabetic mice in a dose-dependent fashion. J Immunol 171:2270-2278). In six replicates, 1.0×10⁵ total cells were used in each of the following Treg:Teff ratios: 2:1, 1:1, and 0.5:1. To each well, 1 μg anti-CD3 antibody and 1 μg anti-CD28 antibody was added. In addition to the combinations of Treg and Teff cells, accessory cells irradiated with 3300 rads were added to each well. In other wells, accessory cells were plated alone both with and without lag anti-CD3/1 μg anti-CD28 antibody. The cells were then incubated at 37° C., 5% CO2, 95% humidity for 5 d. On d 4, 0.5 μCi H3 thymidine was added to each well. Following 18 h of incubation, the cells were lysed and the H3 incorporation determined using a 1450 Microbeta Trilux®β-scintillation counter (Wallac, Turku, Finland).

Flow cytometry. Spleen, DC, BM, ILN, and PLN were collected (as noted) from mice and subjected to flow cytometric analysis using either a FACScan® or FACScalibur flow cytometer (Becton Dickinson). Flow cytometric analysis of prepared cells was performed with 5.0×10⁴ to 1.0×10⁵ cells from each sample. Data were analyzed using the FCS Express® analysis program (De Novo Software, Thornhill, Canada). For each mouse, cells were labeled using antibodies (as well as relevant isotype controls) purchased with one exception, from a single commercial vendor (BD Pharmingen) including: anti-CD3, CD4, CD8, CD11b, CD25, CD28, CD86, CD154, as well as anti-MHC class II. Anti-Foxp3 antibody was purchased from a second vendor (eBiosciences). In all situations, antibodies were utilized according to manufacturers recommendations.

Adoptive transfer. Splenocytes were obtained from NOD mice of various treatment groups at 30 week of age and adoptively transferred or co-transferred via intravenous injection into NOD.rag^{-,-} mice. Mice receiving 2.0×10⁶ splenocytes from four untreated mice with recent onset T1D served as a methodological control. For transfer studies, NOD.rag^{-,-} mice received 2.0×10⁶ splenocytes from 12 week rIgG treated mice or 2.0×10⁶ splenocytes from 12 week mATG treated mice. In studies of adoptive co-transfer, NOD.rag^{-,-} mice were provided 1.0×10⁶ splenocytes from untreated recent onset T1D mice mixed with 1.0×10⁶ splenocytes from 12 week rIgG treated or mATG treated mice. All the mice were followed for onset of T1D as described previously.

Statistical analysis. Statistical analysis was performed using Kaplan-Meier life table analysis, one-way ANOVA, or Fisher's Exact (two tailed) testing. All data are presented as mean±SD. P values<0.05 were deemed significant.

### EXAMPLE 1

### Lymphocyte Depletion after Treatment with mATG

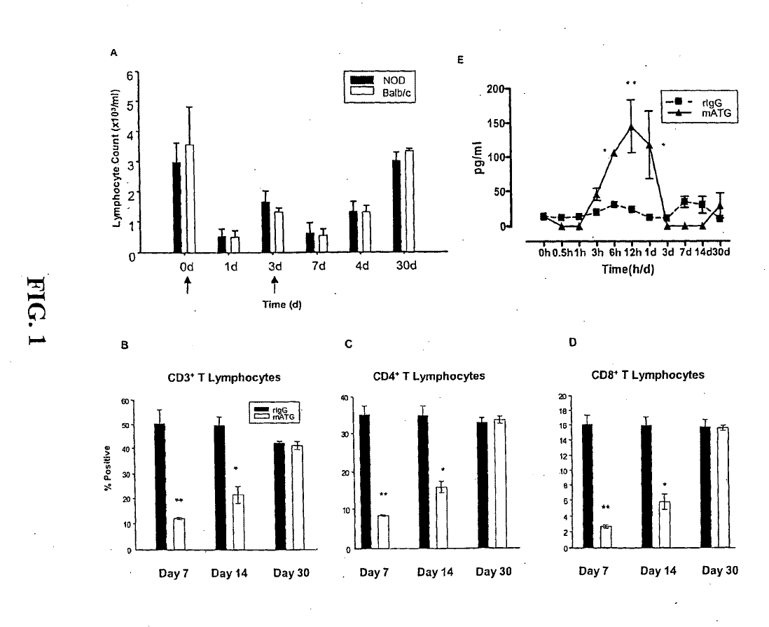

To evaluate whether the in vivo activities of mATG demonstrate strain specific differences in terms of its capacity for lymphocyte depletion, whole blood samples were collected at various times from 4, 8, or 12 week old NOD as well as Balb/C mice both prior to and up to 30 d following intraperitoneal administration of mATG. Treatment of both strains of mice with mATG induced a significant degree of lymphopenia within 1d (FIG. 1 A), but one which by 30 d post-administration, peripheral blood lymphocyte counts returned to pre-administration levels. No significant differences were observed in lymphocyte counts between mATG treated NOD and Balb/c mice at any treatment age (all P=NS); represented by similar patterns of depletion and subsequent restoration over a 30 d period (12 week data; FIG. 1A). Hence, mATG treatment imparts a period of transient lymphocyte depletion followed by a robust recovery of cells, with no age- or strain-dependent variations being noted in terms of either lymphocyte depletion or recovery.

### EXAMPLE 2

### Depletion of CD3⁺, CD4⁺, and CD8⁺ T Lymphocyte Populations is Transient Following mATG Treatment

To identify the actions of mATG on a variety of these T cell subsets and (potentially) uncover any bias in terms of its actions in vivo, flow cytometry was used to evaluate the levels of CD3⁺, CD4⁺, and CD8⁺ T cell populations 7, 14, and 30 d following treatment in NOD mice treated with mATG or rIgG. Treatment of NOD mice with mATG versus rIgG, imparted a transient decline in CD3+ T cells, (12.1±0.8% vs. 53.7±6.8%; respectively; P<0.01) which by 30 d post-administration, returned to pre-administration levels (FIG. 1 B). This pattern was also observed with CD4⁺ (FIG. 1C; 8.5±0.2% vs. 38.4±2.4%) and CD8+(FIG. 1D; 2.6±0.2% vs. 16.1±1.8%) T cell populations. Throughout this 30 d period, the CD4:CD8 ratio of the mATG treated mice was not significantly altered from rIgG treated mice (P=NS), nor were T-lymphocyte subsets of any rIgG treated mice reduced.

### EXAMPLE 3

### Transient Serum Cytokine Increases Follow mATG Treatment

Serum samples from mice were collected at 0, 1, 3, 6, 12, and 24 h as well as 3, 7, 14, and 30 d following treatment of NOD mice provided mATG or rIgG. A 21-plex Luminex® cytokine assessment was performed on each serum sample for cytokine profiling.

mATG imparted a cytokine release pattern in vivo that was marked by transient, but statistically significant increases in many cytokines (IL-1α, IL-1, IL-2, IL-4, IL-5, IL-6, IL-7, IL-9, IL-10, IL-12p70, IL-13, IL-15, IL-17, IFN-γ, TNF-α, GM-CSF, MP-1α, MCP-1, KC, RANTES, IP-10, and G-CSF). Indeed, of these, only G-CSF and IL-12 p70 were not significantly elevated in mATG versus rIgG treated mice. For example, serum IL-2 levels increased in mATG treated mice from an average baseline of less than 20±1.9 to 145±46.1 pg/mL within 12 h, but declined to baseline levels by d 3 (FIG. 1E). NOD mice treated with control rIgG did not exhibit a significant increase in any serum concentration, including IL-2 (FIG. 1E), throughout the measured time points (P=NS). These observations demonstrate another example of the rapid yet transient immunological consequences of mATG treatment in vivo.

### EXAMPLE 4

### A Time Dependent Prevention of T1D is Imparted by mATG Treatment

NOD mice (12 to 18 per group) were provided mATG or rIgG at 4, 8, or 12 week of age. Mice were monitored for T1D development to 30 week of age; however a series of animals from each study group were randomly sacrificed during this time period to provide for additional mechanistic studies. Group sizes were set to nine animals per treatment arm for each age group to assess the influence of these agents on the natural history of the disease.

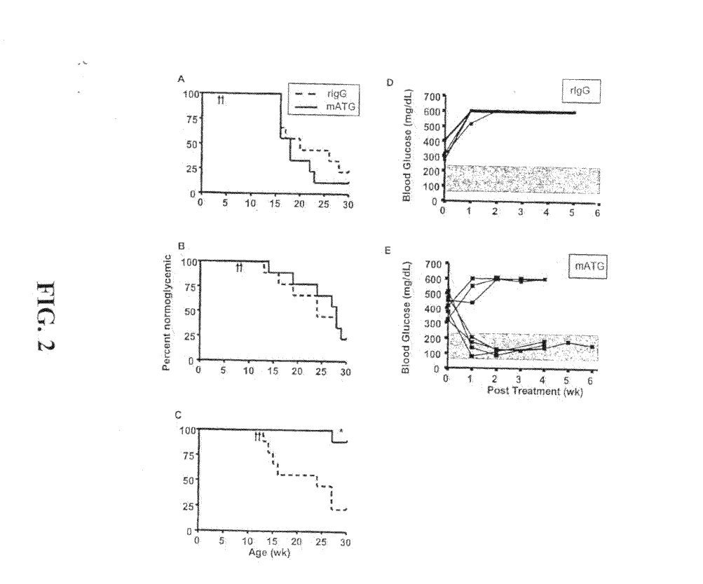

No differences in either the overall frequency or rate in the progression of T1D (i.e., life-table analysis) were seen between NOD mice provided rIgG or mATG at either 4 week or 8 week of age (FIG. 2 A, B; P=NS). In contrast, NOD mice treated with mATG at 12 week of age resulted in a significant decrease in the development of T1D in comparison to rIgG treated littermates (FIG. 2 C). Indeed, at 30 week of age, 89% (8/9) of mATG treated mice remained euglycemic while only 22% (2/9) of rIgG treated mice were without T1D (P=0.015).

### EXAMPLE 5

### A Reversal of Overt Hyperglycemia can be Afforded by mATG Treatment in NOD Mice

The issue of T1D reversal in NOD mice at the overt onset of hyperglycemia was studied. It should be emphasized that in our efforts involving mATG, no exogenous insulin replacement was provided to these animals in order to provide an exceptionally rigorous measure of therapeutic efficacy. Utilizing the same treatment schedule for disease prevention (i.e., 1.0 mg/animal, 72 hours apart), mATG was observed to provide a significant degree of disease reversal versus administration of rIgG (4/7 (57%) vs 0/6 (0%), repetitively; P=0.05) (FIGS. 2D and 2E).

Taken collectively with the studies of disease prevention, the therapeutic benefits afforded by mATG treatment demonstrated a clear age dependence; suggesting that additional factors related to the stage in the natural history of T1D development were associated with the ability to prevent disease. Among the mechanisms that could underlie this observation are those related to the local influences (both qualitative and quantitative influences) of mATG on the insulitis lesion or the pancreatic lymph node, as well as systemic influences activating components promoting immunoregulatory mechanisms affording islet cell protection.

### EXAMPLE 6

### mATG Treatment Attenuates Insulitis and Improves Response to Glucose Levels in Response to Metabolic Challenge

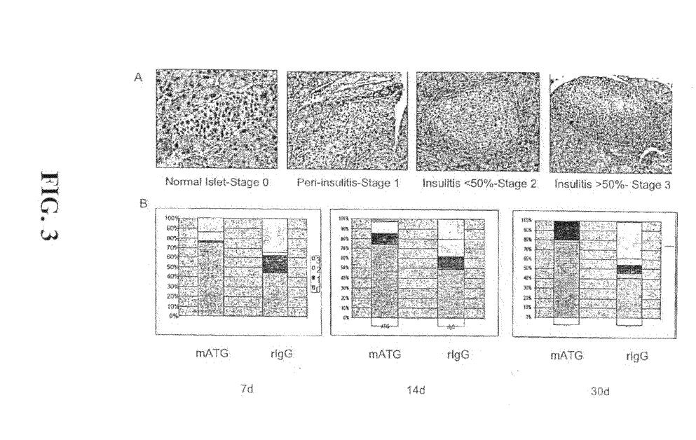

To determine the nature of protection from T1D onset afforded by mATG treatment at 12 week of age, the degree of pancreatic insulitis following treatment was assessed. As shown in FIG. 3, mice treated with mATG at 12 week of age exhibited significantly lower levels of infiltration in comparison to rIgG treated animals. Interestingly, this pattern demonstrating a less severe form of insulitis increased over time, suggesting above and beyond the initial depletion afforded by mATG, the agent may induce protective mechanism(s) attenuating in migration of cells to the pancreatic islets.

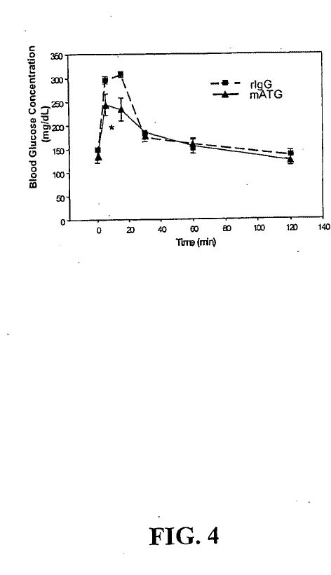

Further confirmation of attenuated autoimmunity in the group treated with mATG at 12 week of age was observed in intraparitoneal glucose tolerance tests. Thirty days after the first injection, mATG or rIgG treated mice underwent intraperitoneal glucose tolerance testing (IPGTT). Blood glucose values were obtained at 0, 5, 15, 30, 60, and 120 min following glucose administration (FIG. 4). Glucose levels were not significantly different in mice treated with mATG at 4 and 8 week of age compared with that of rIgG recipient mice. In contrast, mice treated with mATG at 12 week of age demonstrated a significant divergence between the mATG and control rIgG treated mice (P<0.05), by area under the curve analysis (AUC), following glucose administration (FIG. 4). While mice from both treatment groups had similar fasting glucose levels, upon glucose administration, rIgG treated mice average glucose levels rose to a peak of 307±5.0 mg/dL while mATG treated mice glucose levels only elevated to 244.7±22.8 mg/dL, demonstrating a more severe impairment in glucose response in rIgG mice (P<0.02). These metabolic findings noting improved function in mATG treated mice, combined with their reduced levels of insulitis, shows an age specific attention of beta cell autoimmunity and preservation of the capacity for insulin secretion.

### EXAMPLE 7

### Flow Cytometric Analysis of Antigen Presenting Cell and Regulatory T Cell Populations In Vivo Following mATG Treatment

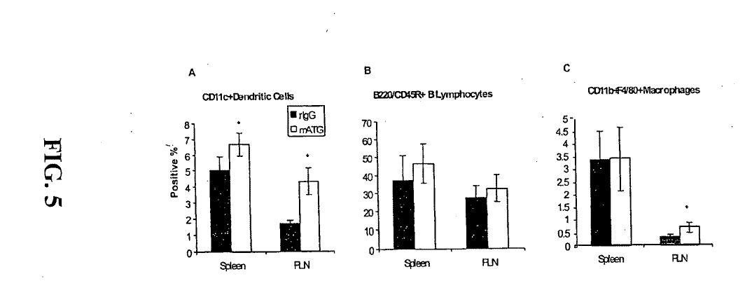

The levels of various antigen presenting cells (APC) including dendritic cells (DC), B-lymphocytes, and macrophages following mATG or rIgG treatment were evaluated. In addition, based on the pivotal role DC play in the generation of T cell responses, the impact of mATG treatment on the profile and function of DC were examined in order to elucidate potential mechanisms underlying the disease protection observed with this agent. To perform such assessments, NOD mice at 12 week of age were injected intraperitoneally with mATG or polyclonal rabbit IgG as control, and sacrificed 24 h later (FIG. 5). Spleen, pancreatic lymph node (PLN), inguinal lymph node (ILN), and bone marrow (BM) cells were harvested for flow cytometric analysis of markers associated with the aforementioned cell populations (FIG. 5).

mATG treatment increased the frequency of DC (number of DC/total cells) in a number of lymphoid tissues, including spleen (rIgG vs mATG; 5.0%±0.88% vs 6.7%±0.71%; P<0.01), PLN (1.6%±0.22% vs 4.3%±0.81%; P<0.01) and ILN (1.7%±0.27% vs 4.5%±0.85%; P<0.01), but not in thymus or BM (P=NS). The largest increase occurred in the CD11b⁺CD8⁻PDCA-1⁻ DC subtype. Although this increase may reflect the relative reduction in T cell number associated with mATG, the frequency of CD8⁺DC⁻ among total DC was markedly reduced in spleen (28%±4.5% vs 17%±2.8%; P<0.01), PLN (34%±3.1% vs 16%±3.7%; P<0.01), ILN (58%±8.5% vs 42%±8.0%; P<0.01) and BM (20%±2.1% vs 6.7%±0.7%; P<0.01). Relative to rIgG treated animals, DC from mice receiving mATG exhibited a more mature phenotype (CD86^{hi}MHC II^{hi}) in spleen (44%±5.7% vs 57%±5.6%; P <0.01), PLN (58%±4.9% vs 78%±1.2%; P<0.01), ILN (65%±7.5% vs 84%±5.8%; P<0.01) and BM (16%±4.4% vs 31%±5.3%; P<0.01). CCR7 expression was also upregulated on DC from spleen (8.9%±0.30% vs 15%±2.4%,P<0.01) and PLN (17%±3.9% vs 43%±17%). The frequency of CD4⁺CD25⁺Foxp3⁺ T cells was increased in PLN (7.8%±1.6% vs 15.8%±1.7%; P<0.01) and ILN (7.8%±0.79% vs 15.8%±2.3%; P<0.01) after mATG treatment. Taken collectively, these findings suggesting that mATG treatment promotes DC migration to lymphoid tissues from peripheral organs and that a tolerogenic DC phenotype develops that promotes the attendant expansion of Treg, which likely contribute to T1D prevention.

### EXAMPLE 8

### mATG Treatment Augments CD4⁺CD25⁺ Cell Frequencies

Splenocytes from mATG or control rIgG treated mice were removed and stained at various time points for a markers of cell populations previously associated with the pathogenesis of this disorder. Specifically, flow cytometric analysis revealed increased expression of CD4⁺CD25⁺ cells at 7 d (16.85±2.09% vs. 8.30±0.27%; P<0.01) and at 14 d (11.06±0.23% vs. 8.26±0.27%; P<0.05) post-mATG treatment. In addition, increased levels of CD4⁺CD28⁺ cells (3.58±0.34% vs. 1.12±0.32%; P<0.05) and CD8+CD28+(2.78±0.12% vs. 0.89±0.19%; P<0.01) were observed 7 d post mATG treatment compared to control rIgG treated littermates. Additional analysis of these or other cell populations at time points to 30 d post therapy did not reveal differences in cellular frequencies. Taken collectively, these studies suggest the capacity for mATG to induce a transient imbalance of the frequency of cells favoring regulatory T cell activities in vivo.

### EXAMPLE 9

### mATG Treatment Enhances the Functional Activities of CD4⁺CD25⁺ T Cells

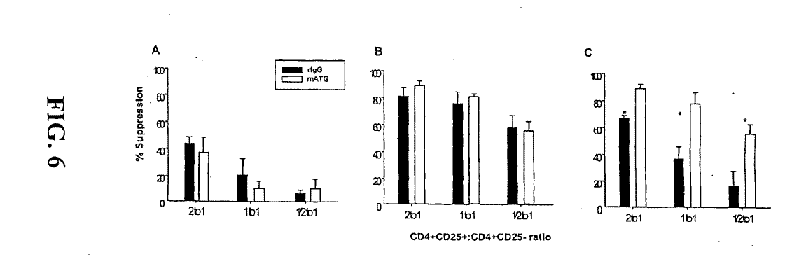

To farther decipher the potential protective mechanisms against T1D development induced by mATG treatment, the capacity for this therapy to modulate Treg cell function was investigated. Towards this end, purified CD4^{+CD}25⁺ T lymphocytes from different experimental groups administered with mATG or rIgG antibodies were mixed with varying ratios to effector CD4⁺CD25⁻ T lymphocytes, and proliferation following anti-CD3/CD28 stimulation determined (FIG. 6). Mice treated. with mATG at 4 week of age and sacrificed at 30 d demonstrated a reduced (albeit not statistically significant) ability to suppress effector T cell proliferation (FIG. 6A). Mice treated with mATG at 8 week of age showed an equivalent capacity to suppress stimulated effector T cells (FIG. 6B), in comparison to rIgG. In contrast, mice treated with mATG at 12 week of age demonstrated a marked decrease in average proliferation of effector CD4⁺ cells in the presence of regulatory T cells at a 2:1, 1:1, and ½:1 ratios. Indeed, the largest difference in this capacity was seen at 1:I ratio in which CD4⁺CD25⁺ T lymphocytes from mice treated with mATG suppressed lymphocyte proliferation by 78%±8.2 (P<0.01), as compared to 37.3%±8.2 suppression with CD4⁺CD25⁺ T lymphocytes purified from mice treated with rIgG (FIG. 6C). Therefore, much like the observations involving T1D prevention suggesting age dependencies, mATG treatment appears to augment Treg function in vivo in a more limited time frame (i.e., 12 week of age).

### EXAMPLE 10

### mATG Treatment Alters Diabetogenic and Immunomodulatory Activities In Vivo

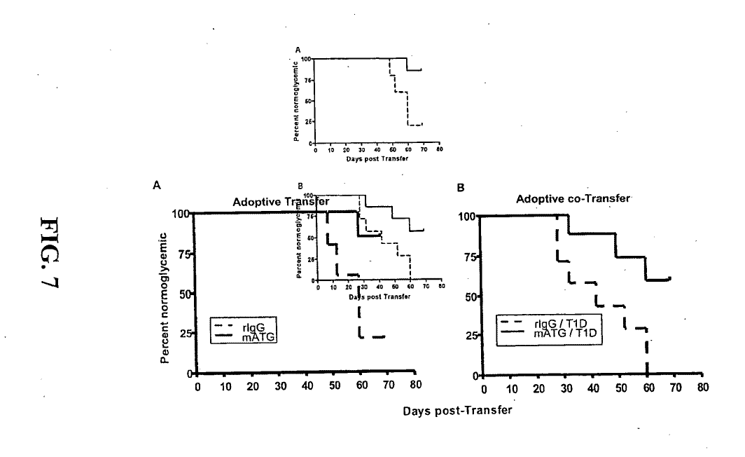

To further characterize the potential of this treatment to impart a degree of immunoregulation capable of attenuating anti-O cell immunity, as well as to establish whether mATG altered the innate capacity for treated mice to develop T1D, both adoptive transfer and adoptive co-transfer studies were performed. For studies of adoptive transfer, splenocytes were obtained at 30 week from non-diabetic survivors that were mATG or rIgG treated at 12 week of age and administered via intravenous tail vein injection into to NOD.rag^{-,-} mice (FIG. 7). In animals subject to this procedure, T1D onset was delayed (FIG. 9A; P<0.03) and occurred at a reduced frequency [17% (1/6) versus 80% (4/5)] in mice that received mATG versus rIgG, respectively.

In a parallel set of adoptive co-transfer studies, 10×10⁶ splenocytes from 30 week old mATG or rIgG treated mice were mixed at a 1:1 ratio with 1.0×10⁶ splenocytes obtained from a set of untreated NOD mice with recent-onset T1D and transferred into NOD.rag^{-/-} mice. Similar to the observations involving adoptive transfer, co-transfer of 2.0×10⁶ splenocytes representing the mixture from 30 week old mATG treated mice into NOD.rag⁴ mice modulated the degree of diabetes development not observed with co-transfers with cells from rIgG treated animals (FIG. 7B; P<0.02). These in vivo data support the aforementioned in vitro data suggesting that mATG induces cells capable of attenuating autoreactive effector T cells.

### EXAMPLE 11

### Animal Model

NOD/LtJ mice were purchased from Jackson Laboratories and arrived at 8 weeks of age. Diabetes onset was typically observed from 12 weeks on.

### Husbandry

Animals were kept in sterile microisolator cages with sterile bedding and given free access to sterile food and acidified water (pH≅2).

### Blood Glucose Monitoring

Blood glucose levels were determined twice weekly in animals starting at 10 weeks of age using a handheld glucometer. Blood samples were collected by tail nick at approximately the same time of day for the duration of the study. Animals were treated with insulin an pellet and assigned to treatment group when blood glucose reading≧300 mg/dL.

### Insulin Pellet Administration

The animal was briefly anesthetized with isoflurane, approximately 2 cm² section of skin on the back was shaved and the site cleansed with an iodine solution and an ethanol solution. Insulin pellets from LinShin Canada were administered subcutaneously using a trocar. Glucose homeostasis was maintained for approximately 3-4 weeks.

### Mouse Anti-Thymocyte Globulin Administration

Two doses of anti-thymocyte globulin (ATG) were intraperitoneally delivered 72 hours apart. Individual animals were given each of the two doses at the same concentration. The dose cohorts ranged from 150 to 500 μg/dose (which correlated to approximately 6 to 625 mg/kg body weight). Each animal was pretreated with an intraperitoneal administration of dexamethasone (2 mg/kg) two hours prior to receiving anti-thymocyte globulin (ATG).

### End Point Analysis

Maintenance of blood glucose homeostasis (measured in days) in the absence of exogenous insulin was determined for each animal. Two to three consecutive blood glucose measurements in excess of 600 mg/dL signified hyperglycemia and the animals were euthanized.

### Summary

Reversal of type 1 diabetes, as defined by blood glucose homeostasis in the absence of exogenous insulin administration, was observed in 50 to 70% of the NOD mice treated with ATG. The efficacious ATG doses ranged from 200 to 500 μg/dose (which correlated to approximately 8 to 625 mg/kg body weight).

Accordingly, doses of about 5 to 750 mg/kg can be used. Doses from about 50 to 500 mg/kg and 100 to 250 can be used. Multiple doses can be used over, for example 72 to 96 hours.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A method to prevent or delay the onset of the clinical manifestation of type 1 diabetes wherein said method comprises administering, to a person at risk for developing type 1 diabetes, anti-thymocyte globulin (ATG).

2. The method, according to claim 1, wherein said method further comprises administering a compound that promotes the repair, production, and/or regeneration of beta cells.

3. The method, according to claim 2, wherein said compound that promotes the repair, production, and/or regeneration of beta cells is selected from the group consisting of glulisine, glucagons, DPP4 inhibitors, islet regeneration molecules, anti-apoptotic molecules and exendin-4.

4. The method, according to claim 3, wherein the glucagon is glucagon-like peptide-1 (GLP-1).

5. The method, according to claim 1, wherein ATG is administered to a patient who has at least one of the following: islet cell antibodies (ICA), insulin autoantibodies (IAA), glutamic acid decarboxylase antibodies (GADA), or insulinoma-associated-2-autoantibodies (IA-2A).

6. The method, according to claim 5, wherein the patient has at least two of the listed antibodies.

7. The method, according to claim 1, wherein the patient has been determined to have a decreased first-phase insulin response to the administration of intravenous glucose.

8. The method, according to claim 1, wherein beta cell mass has declined to less than 50% but more than 10% of normal.

9. The method, according to claim 1, wherein the patient is genetically pre-disposed to type 1 diabetes.

10. A method for enhancing the ability of CD4+CD25+ T cells to defend against pathological autoimmune processes, wherein said method comprises administering, to a patient in need of an enhanced defense against pathological autoimmune processes, anti-thymocyte globulin (ATG).

11. The method, according to claim 10, which is used to prevent or delay the progression of a condition selected from the group consisting of type 1 diabetes, rheumatoid arthritis, multiple sclerosis, thyroiditis, inflammatory bowel disease, Addison's disease, pancreas transplantation, kidney transplantation, islet transplantation, heart transplantation, lung transplantation, and liver transplantation.

12. A method for reversing type 1 diabetes wherein said method comprises administering, to a person in need of such treatment, anti-thymocyte globulin (ATG).

13. The method, according to claim 12, which comprises administering approximately 8 to 625 mg/kg body weight of ATG.

14. The method, according to claim 12, wherein multiple doses of ATG are administered.

15. The method, according to claim 12, wherein approximately 8 to 625 mg/kg of ATG are administered over a period of 72 to 96 hours.

16. Use of anti-thymocyte globulin (ATG) in the manufacture of a medicament for the prevention or reversal of type 1 diabetes.
